(19) United States
(12) Patent Application Publication
(10) Pub. No.: US 2003/0215491 A1
(43) Pub. Date: Nov. 20, 2003

(54) Method and composition for rapid delivery of bioactive compounds to the systemic circulation via the nasal membrane

(76) Inventors: Charles B. Hensley, Redondo Beach, CA (US); Robert S. Davidson, Woodland Hills, CA (US)

Correspondence Address: PERKINS COIE LLP, POST OFFICE BOX 1208, SEATTLE, WA, 98111-1208, US

(21) Appl. No.: 10/258,978

(22) PCT Filed: Apr. 30, 2001
(86) PCT No.: PCT/US01/13973

(51) Int. Cl. A61K 9/127; A61K 31/355; A61K 31/716; A61K 31/734; A61K 33/04; A61K 35/78; A61K 31/375

(52) U.S. Cl. 424/450; 514/55; 514/60; 514/560; 514/474; 514/54; 514/458; 424/702; 424/729; 424/94.4; 514/2

(57) ABSTRACT

A bioadhesive phosphomatrix composition and method for rapid delivery of effective amounts of active substances through the nasal membrane into the circulatory system. The composition may contain permeation enhancers that facilitate delivery of the active substance to the systemic circulation.

### FIELD OF THE INVENTION

The present invention relates to the field of drug delivery across a nasal mucous membrane.

### BACKGROUND OF THE INVENTION

The current techniques for systemic drug delivery have one or more deficiencies relating to either the effectiveness or the convenience of the techniques.

Subcutaneous injection is an inefficient means of getting the drug into the circulatory system and is therefore a poor method when rapid distribution is required. Additionally, subcutaneous injection is painful, leading to compliance and accessibility issues, and syringes require a prescription. Thus subcutaneous injection is a poor method of administering drug outside of a clinical environment. These arguments are even more appropriate when applied to the deficiencies of intravenous administration.

Transdermal delivery systems offer substantial benefits in case of administration versus the aforementioned invasive techniques, although transdermal delivery is impracticable for many drugs due to their size and polarity. Additionally, precise dosing with a transdermal patch is difficult due to the varying diffusion environment as a function of body-fat, skin thickness and local conductivity.

In principal, methods of diffusing a drug across the nasal mucosa offer substantial benefits over other methods of systemic drug administration including the efficient uptake into venus circulation because of the extensive vascularization surrounding the nasal mucosa and the relatively permeable nasal mucosa membranes. Although a number of techniques to date have attempted to administer drugs, mostly small metal cations, such as zinc, via the nasal mucosal membranes, these techniques have generally been ineffective at achieving pharmacologically effective, systemic drug levels of larger more complex biologically active molecules, such as peptides, liposomes, nucleic acids, or other high molecular weight organic molecules. See, e.g., U.S. Pat. No. 6,080,783, U.S. Pat. No. 5,688,532, and U.S. Pat. No. 5,622,724. The aforementioned patents disclose and claim compositions for delivering pharmacologically effective amounts of zinc ions across the nasal mucosal layer comprising: an aqueous or polar carrier and an effective amount of zinc. As one skilled in the art would appreciate, aqueous or polar carriers are substantially adapted for solvating a small cationic species, like a zinc ion. They are not adapted for delivering large organic molecules, and consequently would be inefficient carriers of such.

Accordingly, an object of this invention is a composition for systemic delivery of a drug or other biologically active molecule across the nasal mucosal membranes. Another object of this invention is a method systemically delivering a drug or other biologically active molecule across the nasal mucosal membrane. The compositions and methods according to this invention are based on the realization that a drug or other biologically active molecule may be easily diffused into the circulatory system across the nasal mucosal membrane. Additionally, the compositions and methods of this invention are based on the realization that the rate of diffusion across the nasal mucosal membrane may be increased if the drug to be diffused is first solvated in an environment with comparable polarity to the phospholipid membranes comprising the nasal mucosal membranes.

### SUMMARY OF INVENTION

One aspect of the invention is a phosphomatrix composition used for systemically administering a drug or a biologically active molecule via the nasal mucosal membrane comprising: a phosphomatrix carrier and a biologically active compound. Another aspect of the invention is a method of administering a drug or a biologically active molecule by contacting a preferred phosphomatrix composition containing a drug or a biologically active molecule with the nasal mucosal membrane for a sufficient period of time to administer a pharmacologically effective dose of the drug or biologically active molecule.

### DETAILED DESCRIPTION OF THE PREFERRED EMBODIMENTS

This invention relates to a composition and a method for systematically administering a drug or a biologically active molecule via the nasal mucosal membrane.

A preferred aspect of the invention is a phosphomatrix composition for systematically administering a drug or a biologically active molecule via the nasal mucosal membrane, comprising a phosphomatrix carrier and a drug or a biologically active molecule. A preferred phosphomatrix carrier preferably comprises one or more phospholipids selected from the group consisting of: phosphatidylcholine, phosphatidylethanolamine, phosphatidylserine or phosphatidylinositol. The phosphomatrix carrier may also include a liquid component selected from the group of water, oils and fatty acids. This liquid component of the phosphomatrix carrier preferably varies in concentration from about 0-90% by weight. Suitable oils include polyunsaturated oils and monounsaturated oils, such as omega 3 and omega 6, and DHA. The preferred compositions and methods according to this invention are based on the realization that the epithelial cells comprising the nasal mucosal membranes are composed of similar phospholipids. Accordingly, in addition to these preferred phospholipids, one skilled in the art will appreciate that any amphiphilic fatty acid or other high molecular weight hydrocarbons of similar polarity to membrane phospholipids may be employed in the phosphomatrix carrier.

The phosphomatrix composition may also include adhesives such as carageenan, methyl cellulose, and guar gum. These adhesives not only increase the density of the carrier, but also help bind the carrier and increase its frictional coefficient to contact the mucosal membranes. Adhesives may comprise from about 0.00001 to about 5% by weight of the phosphomatrix composition. The phosphomatrix composition may also include permeation enhancers for encapsulating a drug or other biologically active molecule such as liposomes, chitosan microparticles and starch micro spheres.

As used herein, a “permeation enhancer” functions to facilitate the passage of an active substance through the nasal membrane, to protect an active substance from being damaged, degraded or altered as it passes through the nasal membrane, and/or to carry an active substance to a desired target in the body after the active substances passes through the nasal membrane. Examples of membrane permeation enhancers include liposomes, chitosan microparticles, and starch microspheres. A liposome, chitosan microparticle, or starch microsphere can encapsulate a drug or other active substance and can protect the drug from damage or alteration as the encapsulation vehicle passes from the nasal cavity, across the epithelial cell, and into the systemic circulation. The liposomes, chitosan microparticles, or starch microspheres also facilitate passage through the nasal epithelial membrane by entering, passing through, and exiting a cell which comprises a portion of the nasal membrane.

Liposomes are spheres of phospholipid which encapsulate the deliverable contents. Liposomes effectively protect the encapsulated content and due to their phospholipid composition, are able to pass across epithelial barriers into the blood stream.

A liposome can be constructed to be a stealth liposome, which is less “detectable” by the liver and thus less subject to elimination by the liver. For example, incorporation of polyethylene glycol moieties into a liposome can give the liposome “stealth” properties. The liposome may be used to target its contents to a specific site in the body. For example, antibodies produced from proximal tubule antigen can be attached to the liposome carrying the active substance. When this “immuno-liposome” enters the systemic circulation, it will have an affinity towards the proximal tubule and will tend to accumulate in the renal tissue.

Permeation enhancers can, by enlarging or loosening tight junctions between cells in the nasal membrane, facilitate the passage of an active substance, a liposome, or another permeation enhancer through the nasal membrane. By way of example, and not limitation, EDTA can chelate calcium. By removing calcium from the cell junctions, EDTA may loosen up the junctions to facilitate passage of an active substance, liposome, etc. through the junction. Liquid permeation enhancers include poly-L-Arg, ascorbic acid, glycerol, and lysophosphatidylcholine. Permeation enhancers may range in concentration from about 0.00001% to about 5% by weight of the phosphomatrix composition.

Protease inhibitors may also be employed as permeation enhancers since they inhibit the proteases in the nasal cavity that could degrade the drug or biologically active molecule to be delivered.

Antioxidants, such as mixed tocopherols, green tea catechin, epigallate, superoxide dismutase (“SOD”) and selenium may also be employed as permeation enhancers.

Emulsifiers such as glycerol may also be employed in the phosphomatrix composition, especially if the phosphomatrix carrier has an aqueous compound. Glycerol is an example of an emulsifier because it helps to combine oil with water and to protect the membrane by moisturizing it. Preferred emulsifier concentrations range from about 0.00001% to about 5% by weight of the phosphomatrix carrier.

As used herein, a substance is deemed a drug or a biologically active molecule if it produces a biological effect on the body. As would be appreciated by those of skill in the art, a biological active molecule includes drugs, but also includes gene vectors, nucleic acids, synthetic genes, gene expression cassettes or any factor which interacts with transcription, translation, metabolism or cellular or tissue level function.

A preferred phosphomatrix composition comprises about 75% to about 99.999% by weight of the phosphomatrix carrier and a sufficient amount of a biologically active molecule to produce a pharmacologically effective dose. Typically a phosphomatrix composition comprises about 0.0000001% to about 25% by weight in the composition of the active substance.

A phosphomatrix composition may range in viscosity from about 0.00001 centipoise to about 10,000 centipoise. The viscosity of the phosphomatrix composition may be adjusted by the addition or removal of low viscosity liquid components such as fatty acids, oils or water to the phosphomatrix carrier. As one skilled in the art will appreciate, as the molecular weight of the biologically active molecule increases, the viscosity of the phosphomatrix composition should be decreased to maintain a given diffusion rate. Additionally, depending upon the polarity of the drug to be delivered the rate of diffusion may be adjusted by adjusting the polarity of the phosphomatrix composition by varying the type and amounts of phospholipids and/or liquid components employed in the phosphomatrix carrier.

If small lipophilic molecules that easily diffuse across the epithelial layer are to be delivered, it may not be necessary to employ permeation enhancing liposomes. If larger biologically active molecules and especially larger polar biologically active molecules such as nucleic acids, amino acids or peptides are to be delivered, liposomes are preferably employed. In addition, increasing the rate of diffusion, liposomes or other permeation enhances may also be employed for targeting and protecting the biologically active molecules from degradation or destruction from cellular oxidases, proteases, lipases or even circulating antibodies.

The rate and extent of diffusion of a biologically active molecule across the nasal mucosal membranes and thus the rate at which pharmacologically effective dosages are achieved is a function of the size and polarity of the biologically active molecule, the viscosity and polarity of the phosphomatrix. As a general rule, the rate of diffusion for a biologically active molecule decreases with increasing polarity, increasing molecular weight or increasing phosphomatrix viscosity. As a general rule, the rate of diffusion for a biologically active molecule increases with increasing lipophilicity, decreasing molecular weight and decreasing phosphomatrix composition viscosity. As a further general rule, the rate of diffusion of the biologically active molecule increases as the surface area of the phosphomatrix composition increases. As a further general rule, it is preferable to apply a thinner phosphomatrix composition to the nasal mucosal membranes in order to minimize the effects diffusion gradients. As a further general rule it is also preferable to employ a higher concentration of the biologically active molecule in order to increase the rate of diffusion across the nasal mucosal membranes. As a further general rule, the dosage of biologically active molecule delivered increases with the time the phosphomatrix drug composition is applied to the nasal mucosal membranes.

Another aspect of the present invention is a method of systemically delivering a drug or a biologically active molecule via the nasal mucosal layer. In a preferred embodiment of the invention, a phosphomatrix composition comprising a phosphomatrix carrier and a drug or biologically active molecule is topically applied to the mucosal surface in one or both nostrils. The phosphomatrix may include permeation enhancers, emulsifiers, adhesives and liquid components such as fatty acids, oils or water. The phosphomatrix composition according to this aspect of the invention may range from about 0.00001 centipoise to about 10,000 centipoise and contain from about 0.000001% to about 25% by weight of one or more biologically active molecules. Since the viscosity of this invention ranges over nine orders of magnitude, the phosphomatrix composition may be topically applied to the nasal mucosal membranes as an aerosol, a liquid or gel.

The following examples depict the presently preferred embodiments of the invention for purposes of illustrating the practice thereof and not by way of limitation of the scope of the invention. In the examples, all proportions are by weight and are approximations, unless otherwise noted.

### EXAMPLE 1

One liter of a preferred phosphomatrix composition may be prepared by combining one or more phospholipids selected from the group consisting of phosphatidylcholine, phosphatidylethanolamine, phosphatidylserine, phosphatidylinositol, in combination with glycerin, and a biologically active molecule according to the following proportions:

| Component | Weight Percent |
|---|---|
| MIXED MEMBRANE PHOSPHOLIPIDS | 96.5 |
| GLYCERIN U.S.P. | 2.0 |
| BIOLOGICALLY ACTIVE MOLECULE | 1.5 |

### EXAMPLE 2

Two hundred microliters of the phosphomatrix composition of Example 1 is placed in one nasal passage of a healthy twenty-seven year old male Caucasian patient. Two hundred microliters of the phosphomatrix composition of Example 1 is then placed in the other nasal passage of the patient. Consequently, a total of 400 microliters of the compound is placed in the patient's nose. The phosphomatrix composition remains in contact with at least a portion of the nasal epithelial membrane. Within ten minutes, the bioactive compound contained in the phosphomatrix composition cannot be measured in the nasal cavity.

### EXAMPLE 3

One liter of a preferred phosphomatrix composition may be prepared by combining one or more phospholipids selected from the group consisting of phosphatidylcholine, phosphatidylethanolamine, phosphatidylserine, phosphatidylinositol, in combination with glycerin, and liposomes containing insulin according to the following proportions:

| Component | Weight Percent |
|---|---|
| MIXED MEMBRANE PHOSPHOLIPIDS | 96.5 |
| GLYCERIN U.S.P. | 2.0 |
| LIPOSOMES CONTAINING INSULIN | 1.5 |
| (carrier for insulin) | |

### EXAMPLE 4

Two hundred microliters of the phosphomatrix composition of Example 3 is placed in one nasal passage of a healthy forty-nine year old female African American patient. A portion of the phosphomatrix composition contacts a portion of the nasal epithelial membrane. The phosphomatrix composition remains in contact with at least a portion of the nasal epithelial membrane. After ten minutes the insulin cannot be measured in the nasal cavity.

### EXAMPLE 5

One liter of a phosphomatrix composition is prepared by mixing together one or more mixed membrane phospholipids selected from the group consisting of phosphatidylcholine, phosphatidylethanolamine, phosphatidylserine, phosphatidylinositol, in combination with an Omega 6 fatty acid, glycerin and liposomes containing alpha melanocyte stimulating hormone (α-MSH) according to the following proportions:

| Component | Weight Percent |
|---|---|
| MIXED MEMBRANE PHOSPHOLIPIDS | 87.0 |
| OMEGA 6 (MONOSATURATED OIL) | 10.0 |
| GLYCERIN U.S.P. | 2.0 |
| LIPOSOMES CONTAINING α-MSH | 1.0 |

### EXAMPLE 6

Three hundred microliters of the phosphomatrix of Example 5 is placed in one nasal passage of a healthy twenty-two year old female Japanese patient. A portion of the phosphomatrix composition contacts at least a portion of the nasal epithelial membrane. The phosphomatrix composition remains in contact with at least a portion of the nasal epithelial membrane. After ten minutes, the α-MSH cannot be measured in the nasal cavity.

### EXAMPLE 7

One liter of a phosphomatrix composition is prepared by mixing together one or more membrane phospholipids selected from the group consisting of phosphatidylcholine, phosphatidylethanolamine, phosphatidylserine, phosphatidylinositol, in combination with glycerin, a biologically active molecule, and SOD. The phosphomatrix composition includes:

| Component | Weight Percent |
|---|---|
| MIXED MEMBRANE PHOSPHOLIPIDS | 95.25 |
| GLYCERIN U.S.P. | 2.0 |
| BIOLOGICALLY ACTIVE MOLECULE | 2.25 |
| SOD | 0.5 |

SOD is an antioxidant which may be employed to protect the nasal epithelial membrane from damage due to the irritation caused by certain bioactive compounds.

### EXAMPLE 8

Example 2 is repeated utilizing the phosphomatrix composition of Example 7 in place of the phosphomatrix composition of Example 1. Similar results are obtained.

### EXAMPLE 9

Example 3 is repeated, except that concentration of insulin in the nasal cavity is measured at 10, 20 and 30 minutes. Still there is no measurable insulin.

### EXAMPLE 10

Example 9 is repeated, except, in this case, naked insulin peptides are used rather than liposomes containing insulin. The relative concentrations remain the same. Unlike Examples 3 and 9, the concentration of insulin in the phosphomatrix is reduced more slowly because the rate of insulin diffusion is lower than Examples 3 and 9 because naked insulin peptides do not cross the epithelial barrier of the mucosal membrane as efficiently as when contained in liposome vesicles.

Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A composition for systemically delivering a pharmacologically active amount of a biologically active molecule comprising:
a phosphomatrix carrier; and
a biologically active molecule.

2. The composition of claim 1 wherein the phosphomatrix carrier comprises one or more phospholipids selected from the group consisting of phosphatidylcholine, phosphatidylethanolamine, phosphatidylserine and phosphotidylinositol.

3. The composition of claim 1 wherein the phosphomatrix carrier further comprises a liquid component selected from the group consisting of oils, fatty acids, or water.

4. The composition of claim 3 wherein said oil is a mono- or polyunsaturated oil.

5. The composition of claim 1 wherein said viscosity ranges from about 0.00001 centipoise to about 10,000 centipoise.

6. The composition of claim 1 wherein said phosphomatrix carrier comprises about 75% to about 99.9999% by weight of said composition.

7. The composition of claim 1 further comprising a permeation enhancer.

8. The composition of claim 7 wherein said permeation enhancer is selected from the group consisting of liposomes, starch microspheres or chitosan microparticles.

9. The composition of claim 7 wherein said permeation enhancer is selected from the group consisting of EDTA, poly-L-Arg, ascorbic acid, and lysophatidylcholine.

10. The composition of claim 7 wherein said permeation enhancer is an antioxidant selected from the group consisting of mixed tocopherols, green tea catechins, epigallate, SOD and selenium.

11. The composition of claim 7 wherein said permeation enhancer ranges in concentration from about 0.00001% to about 5% by weight of said phosphomatrix composition.

12. The composition of claim 1 further comprising an emulsifier.

13. The composition of claim 12 wherein said emulsifier ranges in concentration from about 0.00001% to about 5% by weight of said composition.

14. The composition of claim 1 further comprising an adhesive selected from the group consisting of carageenan, methyl cellulose and guar gum.

15. The composition of claim 14 wherein said adhesive ranges in concentration from about 0.00001% to about 5%.

16. A composition for the systemic delivery of a drug or a biologically active molecule comprising:
a phosphomatrix carrier;
a biologically active molecule; and
a permeation enhancer.

17. The composition of claim 16 wherein said phosphomatrix carrier is comprised of one or more phospholipids selected from the group consisting of phosphatidylcholine, phosphatidylethanolamine, phosphatidylserine and phosphotidylinositol.

18. The composition of claim 16 wherein said permeation enhancer is selected from the group consisting of liposomes starch microspheres or chitosan microparticles.

19. The composition of claims 17 or 18 wherein said permeation enhancer is further selected from the group consisting of EDTA, poly-L-arg, ascarbic acid and lysophosphatidylcholine.

20. The composition of claims 17 or 18 wherein said permeation enhancer is further selected from the group consisting of tocopherol, green tea catechins, epigallate, sop and selenium.

21. The composition of claim 16 wherein the said composition has a viscosity from about 0.00001 centipoise to about 10,000 centipoise.

22. The composition of claim 16 wherein said phosphomatrix carrier comprises from about 75% to about 99.9999% by weight of said composition.

23. The composition of claim 16 further comprising an emulsifier.

24. The composition of claim 16 wherein said emulsifier concentration ranges from about 0.00001 to about 5% by weight of said composition.

25. The composition of claim 16 further comprising an adhesive selected from the group consisting of carageenan, methylcellulose and guar gum.

26. A composition for systemically delivering α-MSH comprising:
a phosphomatrix carrier, wherein said phosphomatrix carrier comprises one or more phospholipids selected from the group consisting of phosphatidylcholine, phosphatidylethanolamine, phosphatidylserine and phosphotidylinositol;
an omega 6 fatty acid;
glycerin; and
liposomes containing α-MSH.

27. The composition of claim 26 wherein said phosphomatrix carrier comprises from about 75% to about 99.9999% of said composition.

28. A method for systemically delivering a pharmacologically effective amount of a biologically active molecule comprising: topically applying a composition comprising a phosphomatrix carrier and biologically active molecule to the nasal mucosal membrane.

29. The method of claim 28 wherein said phosphomatrix carrier comprises one or more phospholipids selected from the group consisting of phosphatidylcholine, phosphatidylethanolamine, phosphatidylserine and phosphotidylinositol.

30. The method of claim 28 wherein said phosphomatrix carrier further comprises a liquid component selected from the group consisting of oils, fatty acids, or water.

31. The method of claim 28 wherein said composition ranges in viscosity from about 0.00001 centipoise to about 10,000 centipoise.

32. The method of claim 28 wherein said phosphomatrix carrier comprises about 75% to about 99.9999% by weight of said composition.

33. The method of claim 28 said composition further comprises a permeation enhancer.

34. The method of claim 33 wherein said permeation enhancer is selected from the group consisting of liposomes, starch microspheres or chitosan microparticles.

35. The method of claim 33 wherein said permeation enhancer is selected from the group consisting of EDTA, poly-L-Arg, ascorbic acid, and lysophatidylcholine.

36. The method of claim 33 wherein said permeation enhancer is an antioxidant selected from the group consisting of mixed tocopherols, green tea catechins, epigallate, SOD and selenium.

37. The method of claim 33 wherein said permeation enhancer ranges in concentration from about 0.00001% to about 5% by weight of said phosphomatrix composition.

38. The method of claim 28 wherein said composition further comprises an emulsifier.

39. The method of claim 38 wherein said emulsifier ranges in concentration from about 0.00001% to about 5% by weight of said composition.

40. The method of claim 28 wherein said composition further comprises an adhesive selected from the group consisting of carageenan, methyl cellulose and guar gum.

41. The method of claim 40 wherein said adhesive ranges in concentration from about 0.00001% to about 5% by weight of said composition.
